(19) United States
(12) Patent Application Publication
(10) Pub. No.: US 2023/0091173 A1
(43) Pub. Date: Mar. 23, 2023

(54) TUBE FOR A MEDICAL TREATMENT SYSTEM, AND MEDICAL TREATMENT SYSTEM

(71) Applicant: Carl Zeiss Meditec AG, Jena (DE)

(72) Inventor: Andreas Werner, Aalen (DE)

(21) Appl. No.: 17/933,613

(22) Filed: Sep. 20, 2022

(30) Foreign Application Priority Data
Sep. 21, 2021 (DE) 10 2021 124 400.5

(51) Int. Cl. A61F 9/007; A61M 1/00

(52) U.S. Cl. CPC A61F 9/00745; A61M 1/85; A61M 1/71; A61M 2210/0612

(57) ABSTRACT

A tube for a medical treatment system has a first channel for feeding an irrigation fluid from a console of the system to a medical treatment instrument of the system and a second channel for discharging an aspiration fluid from the instrument to the console. The first and the second channels are arranged in parallel. The first channel has a first wall and the second channel has a second wall. A second material of the second wall has a greater hardness than a first material of the first wall. The tube is formed from two individual tube elements for the channels. The tube elements are welded or adhesively bonded in a connection region. The difference between the hardness of the first material and the hardness of the second material is at least 10 ShA and at most 15 ShA.

### CROSS REFERENCE TO RELATED APPLICATION

This application claims priority of German patent application no. 10 2021 124 400.5, filed Sep. 21, 2021, the entire content of which is incorporated herein by reference.

### TECHNICAL FIELD

The disclosure relates to a tube for a medical treatment system, in particular an ophthalmosurgical system for treatment of an eye, wherein the tube has a first channel for feeding an irrigation fluid from a console of the medical treatment system to a medical treatment instrument of the medical treatment system and a second channel for discharging an aspiration fluid from the medical treatment instrument to the console, wherein the first channel and the second channel are arranged in parallel next to each other in a direction of longitudinal extent of the tube, wherein the channels have respective walls connected to each other in a connection region of the tube. The disclosure further relates to a medical treatment system, in particular an ophthalmosurgical system for treatment of an eye, having at least a medical treatment instrument, a console for supplying fluid to the medical treatment instrument in a defined operating mode of the medical treatment system, and a tube for feeding an irrigation fluid from the console to the medical treatment instrument and for discharging an aspiration fluid from the medical treatment instrument to the console.

### BACKGROUND

Medical treatment systems, which have consoles and medical treatment instruments, and tubes for connection, in particular for fluidic connection, of the consoles to the medical treatment instruments are known in the prior art, and so in principle there is no need for separate documentary evidence of such systems. Various surgical techniques are known for example for the treatment of a clouding of the crystalline lens, also known in medicine as cataract. The most widespread technique is phacoemulsification, in which a thin hollow needle is introduced into a capsular bag, in which the crystalline lens is arranged, and is induced to make ultrasonic vibrations.

The lens can be emulsified by means of the vibrating hollow needle, and lens particles released in the process can be aspirated through an aspiration line by means of a pump. In the process, an irrigation fluid is supplied. The lens particles are aspirated, together with the fluid, as aspiration fluid. As soon as the lens has been completely emulsified and removed, a new artificial lens can be inserted into the then empty capsular bag. The treated patient can in this way recover good vision.

An advanced ophthalmosurgical system that has proven particularly suitable for phacoemulsification is disclosed in US 2017/0216093, for example. In this system, two fluid pumps fluidically connected in parallel are used in each case for the irrigation and also for the aspiration. Each of the fluid pumps has a pump chamber, and a drive chamber separated from the pump chamber by means of a preferably elastic partition element. For the intended operation of the fluid pump, the drive chamber is acted upon by a drive fluid whose drive pressure is varied for performing a respective pump stroke. Depending on this, a deflection position of the elastic partition element thus changes, which has a corresponding effect on the pump chamber. The pump chamber is acted upon by the respective treatment fluid, for example the irrigation fluid, the aspiration fluid or the like. The delivery action can then be achieved by suitably controlling an inlet valve and an outlet valve of the fluid pump.

A deflection position of the elastic partition element is detected by means of a deflection position sensor assigned to the respective fluid pump. A control device of the ophthalmosurgical system, in particular of the console, controls the function of the fluid pump at least depending on a sensor signal of the deflection position sensor and on a drive pressure signal supplied by means of a drive pressure sensor. In addition, the control device can, for example, control the inlet valve and the outlet valve.

By alternate actuation of the respective two fluid pumps connected in parallel, a volumetric flow with very little fluctuation can be obtained during a surgical procedure. In this way, an almost constant intraocular pressure can be obtained in the capsular bag. As long as sufficient irrigation fluid can be delivered, the system can also be operated almost without interruption of the flow of irrigation fluid, even during a very protracted surgical procedure.

For the supply of the medical treatment instrument, here for example a handpiece, the medical treatment system generally has a tube which in particular fluidically couples the console to the medical treatment instrument, such that the irrigation fluid can be fed to the medical instrument and the aspiration fluid can be discharged. For this purpose, twin-flow tubes can be provided, in which a respective channel is provided for each of the fluids. The respective channel is closed along its entire circumference. Generally, at least the elements in contact with the treatment fluid are arranged in a separate cassette, which is exchangeably insertable into a cassette holder of the console. Therefore, the console-side attachment of the tube is usually provided on the cassette.

For the defined operating mode, particularly in the case of ophthalmosurgical systems, it is desirable to keep the intraocular pressure, in particular in the capsular bag, as constant as possible by regulating the pressure of the irrigation fluid and the vacuum of the aspiration fluid. For this purpose, it is desirable that the pressure of the treatment fluid be known as precisely as possible, so that correspondingly precise regulation of this pressure can be achieved.

### SUMMARY

It is an object of the disclosure to provide a tube and a medical treatment system in such a way as to achieve improved reliability in the defined operating mode.

This object can, for example, be achieved via a tube for a medical treatment system. The tube includes: a first channel for feeding an irrigation fluid from a console of the medical treatment system to a medical treatment instrument of the medical treatment system; a second channel for discharging an aspiration fluid from the medical treatment instrument to the console; the first channel and the second channel being arranged in parallel next to each other in a direction of longitudinal extent of the tube; the first channel having a first wall including a first material having a first hardness; the second channel having a second wall including a second material having a second hardness; the second hardness being greater than the first hardness; the tube being formed from two individual tube elements including a first tube element for the first channel and a second tube element for the second channel; the first tube element and the second tube element being welded or adhesively bonded in a connection region; and, wherein a difference in hardness between the first hardness and the second hardness is at least 10 ShA and at most 15 ShA.

The aforementioned object can, for example, also be achieved via a medical treatment system, in particular an ophthalmosurgical system for treatment of an eye. The medical treatment system includes: a medical treatment instrument; a console configured to supply fluid to the medical treatment instrument in a defined operating mode of the medical treatment system; a tube for feeding an irrigation fluid from the console to the medical treatment instrument and for discharging an aspiration fluid from the medical treatment instrument to the console; the tube including a first channel for feeding an irrigation fluid from the console to the medical treatment instrument; the tube further including a second channel for discharging the aspiration fluid from the medical treatment instrument to the console; the first channel and the second channel being arranged in parallel next to each other in a direction of longitudinal extent of the tube; the first channel having a first wall including a first material having a first hardness; the second channel having a second wall including a second material having a second hardness; the second hardness being greater than the first hardness; the tube being formed from two individual tube elements including a first tube element for the first channel and a second tube element for the second channel; the first tube element and the second tube element being welded or adhesively bonded in a connection region; and, wherein a difference in hardness between the first hardness and the second hardness is at least 10 ShA and at most 15 ShA.

As regards a tube of the type in question for a medical treatment system, the disclosure proposes in particular that a material of the wall of the second channel has a greater hardness than the material of the wall of the first channel.

As regards a medical treatment system of the type in question, the disclosure proposes in particular that the tube is configured according to the disclosure.

The disclosure is based on, among other things, the concept whereby the functionality and the stability of the medical treatment system as a whole can be improved through the individual choice of the material of the tube in the region of each of the channels. Particularly in the case of ophthalmosurgical systems, it is generally provided that the connection between the console, or a cassette inserted in the console, and the medical treatment instrument, here in particular the handpiece, is effected via a tube with two channels, which is also referred to as a twin tube. A twin tube is useful, since it can be handled by the operator like a single tube and is therefore very user-friendly. However, there are also situations in which an operator requires two individual tubes in order to be able to work using both hands. This may be the case, for example, in an emergency operation such as an anterior vitrectomy following rupture of the capsular bag or when polishing the internal wall of the capsular bag or when aspirating viscoelastic agent. In these cases, the operator has to be able quickly to separate the double tube over a length of approximately 1 meter, so as to be able to use an individual tube for the irrigation fluid and an individual tube for the aspiration fluid.

The tube according to the disclosure has two channels, which are configured as through-openings arranged parallel to each other and alongside each other in the longitudinal direction, wherein one of the channels, for example a first channel, serves to feed the irrigation fluid from the console or the cassette to the handpiece, and the other of the two channels, for example a second channel, serves to guide or lead the aspiration fluid from the handpiece, or from the eye, to the console or to the cassette inserted in the console. The disclosure therefore makes it possible to obtain the lowest possible surge behavior while at the same time achieving a high degree of flexibility and good ergonomics and also good resistance to kinking. The low surge behavior is achieved according to the disclosure by, among other things, the fact that the tube is formed from two individual tube elements for the channels, the tube elements being welded or adhesively bonded in a connection region, wherein a material of relatively great hardness is provided for the material of the wall of the second channel, wherein the difference in hardness between the hardness of the material of the first channel and the hardness of the material of the second channel is at least 10 ShA and at most 15 ShA. The hardness for the material of the second channel can here be chosen, for example, in a range from 65 ShA to 70 ShA. For the material of the wall of the first channel, which is used for the irrigation fluid, a material is preferably used which has, for example, a hardness in the range of approximately 50 ShA to 60 ShA. A tube can thus be obtained which has different hardness properties transversely with respect to the longitudinal direction. In the region of the first channel, which is provided for the irrigation fluid, the hardness is less than in the region of the second channel, which is provided for the aspiration fluid. In particular as regards the second channel provided for the aspiration fluid, the lowest possible surge behavior can therefore be achieved at the same time with acceptable flexibility and ergonomics and with good resistance to kinking. Moreover, the stability of the second channel thus obtained makes it possible to achieve a sufficient flow of the aspiration fluid even with a small internal diameter.

It is of importance that the difference in hardness between the material of the first channel and the material of the second channel is at least 10 ShA and at most 15 ShA. The tube according to the disclosure can therefore be configured such that the difference in hardness is 13 ShA, for example where the hardness of the material of the first channel is 57 ShA and the hardness of the material of the second channel is 70 ShA, It is only within this narrow range of difference in hardness, from 10 ShA to 15 ShA, that it is possible to achieve good maneuverability and a sufficient resistance to kinking of the first channel while at the same time ensuring that the second channel is sufficiently capable of counteracting a surge, with both the first channel and the second channel being provided with a circular internal cross section. There is a constant wall thickness along the entire wall circumference, except for a connection region of the two walls. Neither the first channel nor the second channel requires zones where the stiffness is increased or reduced by a projection or by a cutout on its respective wall. In this embodiment, the respective channel wall is completely circular in cross section. This is advantageous since it permits a uniform flow in the respective channel and does not allow particles to remain caught on projections or cutouts and to act as seeds obstructing a channel. Since neither the first wall nor the second wall has a projection or cutout along its cross section, there is a constant stiffness of the walls along their entire longitudinal extent, and so there is no danger of the walls collapsing. It is thus possible to achieve a high degree of resistance to kinking and safely ensure a uniform flow of the irrigation fluid and the aspiration fluid.

The tube can thus be produced as a one-piece component which simultaneously makes available the channels for the irrigation fluid and the aspiration fluid. The tube can be produced by extrusion, for example, with two different materials being used during the extrusion, depending on the desired degrees of hardness. Of course, the reliability of the medical treatment system can thus also be improved, because the tube in its intended use, which often requires manual guiding of the medical treatment instrument or corresponding guiding by a robot, remains small and compact and therefore easy to handle, while at the same time the configuration according to the disclosure is able to improve the reliability and stability of the tube. Undesired kinking, which can lead to disturbances during use, can thus be reduced in particular.

It is further proposed that the thickness of the wall of the first channel is smaller than the thickness of the wall of the second channel. In this way, it is possible to achieve an increased resistance to kinking for the second channel, even at a high suction pressure. The thickness of the wall of the first channel is preferably chosen such that, despite the different materials, at least in respect of hardness, a stability can largely be achieved that corresponds approximately to the stability of the wall of the second channel guiding the aspiration fluid.

According to a development, it is proposed that the thickness of the wall of the first channel is at least 2.5 mm and/or the thickness of the wall of the second channel is at least 2.6 mm. In this way, a reliable stability can be achieved in particular for common tube sizes.

It proves particularly advantageous if an internal diameter of the first channel is at least 3 mm, and an internal diameter of the second channel is at least 1.1 mm. Particularly for use in an ophthalmosurgical system, suitable flows of fluid can thus be achieved in the tube, such that a defined operating mode can be realized. Depending on requirements, the internal diameters chosen can of course also be greater, for example in order to be able to reduce flow resistances within particularly long tubes. However, in such a case, provision is preferably also made that the respective thickness of the wall of the first and/or second channel is correspondingly increased.

Furthermore, it is proposed that an external diameter of the wall of the first channel is at least 5 mm, preferably approximately 5.5 mm, and an external diameter of the wall of the second channel is at least 4 mm, preferably approximately 4.1 mm. The advantageous external diameters make it possible in particular to use commercially available plug connectors, for example a commercially available Luer connector. In this way, the disclosure can also be easily retrofitted on existing medical treatment systems, without complicated adaptations being needed. However, the use of the disclosure is not restricted thereto.

The difference between the external diameter of the wall of the first channel and the external diameter of the wall of the second channel is preferably in the range of 0.9 mm to 1.6 mm. With a smaller difference between the external diameters, that is, less than 0.9 mm, the resistance of the wall of the second channel to kinking is greatly reduced. It is only with a difference between the external diameters in the range of 0.9 mm to 1.6 mm that it is possible to achieve a high degree of resistance to kinking of the first wall together with a good stability against a surge in the second channel, at the same time with good ergonomics and maneuverability of the tube as a whole.

Altogether, it is possible to avoid the tube having a poorer resistance to kinking as a consequence of the lower hardness of the first channel for guiding the irrigation fluid. By increasing the thickness of this wall, it is even possible that the tube in this respect has at least substantially the same property, such that an almost homogeneous property, in particular with respect to resistance to kinking, can preferably be achieved.

The advantages and effects indicated for the tube according to the disclosure are of course also equally applicable to the medical treatment system equipped with the tube according to the disclosure, and vice versa.

### BRIEF DESCRIPTION OF THE DRAWINGS

The disclosure will now be described with reference to the drawings wherein:

FIG. 1 shows a schematic block diagram of an ophthalmosurgical system according to the disclosure, with a console, and with a handpiece coupled to the console by means of a tube; and,

FIG. 2 shows a schematic sectional view of the tube from FIG. 1.

### DESCRIPTION OF THE PREFERRED EMBODIMENTS

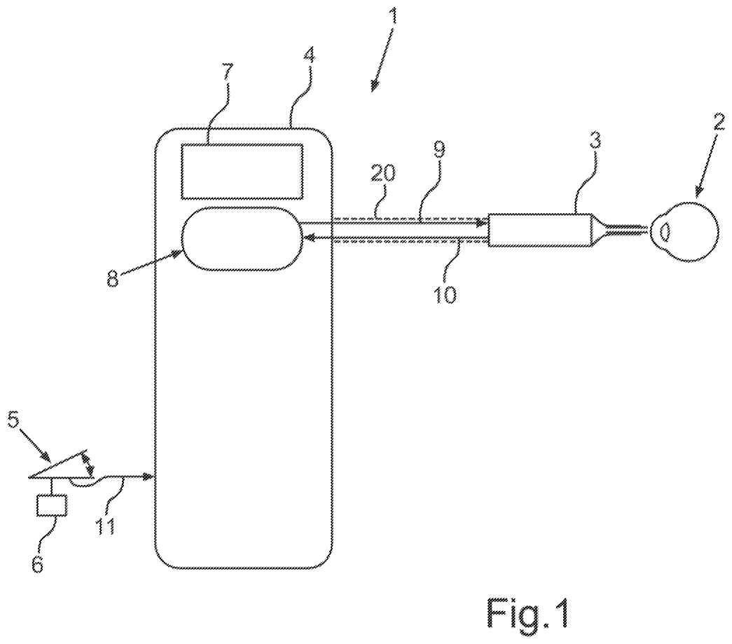

FIG. 1 shows, in a schematic block diagram, an ophthalmosurgical system 1 as a medical treatment system which serves for treatment of an eye 2. The ophthalmosurgical system 1 has a medical treatment instrument, which in the present case is formed by a handpiece 3. Moreover, the ophthalmosurgical system 1 has a console 4 for attachment and operation of the handpiece 3 in a defined operating mode. Furthermore, the ophthalmosurgical system 1 has a foot-operated unit 5, which in the present case is configured as a foot pedal. The foot-operated unit 5 is able to be coupled wirelessly to the console 4 and has an electrical energy store 6 for supplying the foot-operated unit 5 with electrical energy, at least during the defined operating mode of the handpiece 3. Moreover, the console 4 has a control unit 7 and fluidic and electrical supply units 8 which, among other things, serve to feed an irrigation fluid to the handpiece 3 via an irrigation line 9 and to discharge an aspiration fluid from the handpiece 3 via an aspiration line 10.

In the present case, the irrigation line 9 and the aspiration line 10 are made available by a single tube 20, which will be explained in more detail below. The irrigation line 9 is formed by a first channel 24 of the tube 20. The aspiration line 10 is formed by a second channel 25 of the tube 20.

The foot-operated unit 5 in the present case is of a wireless configuration, for which reason it is supplied with electrical energy from the electrical energy store 6, at least during the defined operating mode. In addition, the foot-operated unit 5, in the defined operating mode, communicates with the control unit 7 via a wireless communication link 11, such that an operating state of the handpiece 3 can be at least partially adjusted.

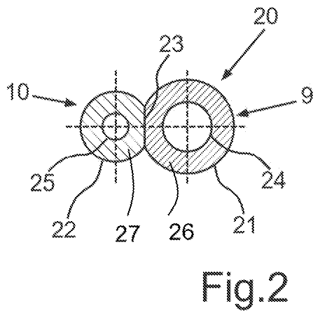

FIG. 2 shows a schematic sectional view of the tube 20 in accordance with FIG. 1. It will be seen from FIG. 2 that the tube has two channels 24, 25, wherein a first channel 24 serves to feed the irrigation fluid from the console 4 of the medical treatment system 1 to the medical treatment instrument 3 of the medical treatment system 1. A second channel 25 serves to discharge the aspiration fluid from the medical treatment instrument 3 to the console 4. The first channel 24 and the second channel 25 are arranged in parallel next to each other, transversely with respect to a direction of longitudinal extent of the tube 20. The tube 20 provides for the first channel 24 a first wall 21 and for the second channel 25 a second wall 22 of predefinable thickness. The walls 21, 22 are connected to each other in a connection region 23. The connection region 23 extends in the present case over approximately the entire length of the tube 20. In an alternative embodiment, the connection region 23 can also extend over only a part of the length of the tube 20.

In the present embodiment, provision is made that a material of the second wall 22 of the second channel 25 has a greater hardness than the material of the first wall 21 of the first channel 24. In the present case, provision is made that the material of the first wall 21 is formed from a polyvinyl chloride (PVC), specifically of type AM157/F 00. In the present case, the material of the second wall 22 is formed by a PVC of type AM171/F.

It is thereby possible that the first wall 21 has a hardness of approximately 57 ShA, whereas the second wall 22 has a hardness of approximately 70 ShA. However, depending on what is needed, the hardness may also be varied, so as to be able to take better account of special requirements.

In order to improve the properties of the tube 20 as regards resistance to kinking, in particular transversely to its longitudinal extent, provision is made here that the thickness of the first wall 21 of the first channel 24 is approximately 2.5 mm. However, depending on requirements, this thickness can also be greater. The thickness of the second wall 22 of the second channel 25 is in the present case approximately 2.6 mm. Here too, the thickness can in some circumstances also be chosen to be greater than this.

The thickness of the first wall 21 of the first channel 24 can also be smaller than the thickness of the second wall 22 of the second channel 25. As a result of the greater thickness of the second wall 21, an increased stiffness can also be achieved in this region, such that a correspondingly increased resistance against kinking can also be achieved.

To be able to make available a sufficient cross section of flow for the defined operating mode of the ophthalmosurgical system, provision is made here that an internal diameter of the first channel 24 is approximately 3 mm. Depending on requirements, this internal diameter can also be chosen to be greater than this. By contrast, in the present case, an internal diameter of the second channel 25 is approximately 1.5 mm. Here too, the internal diameter can also be chosen to be greater than this. It is of importance that the internal diameter of the first channel 24 is always greater than the internal diameter of the second channel 25.

In the present case, an external diameter of the wall 21 of the first channel 24 is approximately 5.5 mm, whereas an external diameter of the wall 22 of the second channel 25 is in the present case approximately 4.1 mm. Depending on requirements, the external diameter of the walls 21, 22 can also be variable, in particular greater.

In the connection region 23, the two walls 21, 22 are cohesively bonded to each other. This can take place, for example, directly during an extrusion of the tube 20. However, the tube 20 can also be formed from two individual tube elements 26, 27 for the channels 24, 25. For example, the tube elements 26, 27 are then not co-extruded but instead extruded individually, after which they are welded or glued to each other. The walls 21, 22 can at least partially overlap in the connection region 23. In this way, the material of the walls 21, 22 can also be used at least in part for connecting the two channels 24, 25 in order to form the tube 20. Individually extruded and then glued or welded tube elements 26, 27 have the advantage that, if need be, the tube elements 26, 27 can later be safely and reliably separated again in the connection region 23 into two individual tube elements 26, 27 and the channels 24, 25 remain intact.

It is understood that the foregoing description is that of the preferred embodiments of the invention and that various changes and modifications may be made thereto without departing from the spirit and scope of the invention as defined in the appended claims.

### LIST OF REFERENCE SIGNS

1 ophthalmosurgical system
2 eye
3 handpiece
4 console
5 foot-operated unit
6 electrical energy store
7 control unit
8 supply unit
9 irrigation line
10 aspiration line
11 wireless communication link
20 tube
21 first wall
22 second wall
23 connection region
24 first channel
25 second channel
26 first tube element
27 second tube element

What is claimed is:

1. A tube for a medical treatment system, the tube comprising:
a first channel for feeding an irrigation fluid from a console of the medical treatment system to a medical treatment instrument of the medical treatment system;
a second channel for discharging an aspiration fluid from the medical treatment instrument to the console;
said first channel and said second channel being arranged in parallel next to each other in a direction of longitudinal extent of the tube;
said first channel having a first wall including a first material having a first hardness;
said second channel having a second wall including a second material having a second hardness;
said second hardness being greater than said first hardness;
the tube being formed from two individual tube elements including a first tube element for said first channel and a second tube element for said second channel;
said first tube element and said second tube element being welded or adhesively bonded in a connection region; and,
wherein a difference in hardness between said first hardness and said second hardness is at least 10 ShA and at most 15 ShA.

2. The tube of claim 1, wherein said first wall of said first channel has a first thickness; said second wall of said second channel has a second thickness; and, said first thickness is smaller than said second thickness.

3. The tube of claim 2, wherein said first thickness is at least 2.5 millimeters.

4. The tube of claim 2, wherein said second thickness is at least 2.6 millimeters.

5. The tube of claim 3, wherein said second thickness is at least 2.6 millimeters.

6. The tube of claim 1, wherein said first channel has a first internal diameter of at least 3 millimeters; and, said second channel has a second internal diameter of at least 1.1 millimeters.

7. The tube of claim 1, wherein said first channel has a first external diameter of at least 5.5 millimeters; said second channel has a second external diameter of at least 4.1 millimeters; and, a difference between said first external diameter and said second external diameter is in a range of 0.9 millimeters to 1.6 millimeters.

8. The tube of claim 1, wherein the medical treatment system is an ophthalmosurgical system for treatment of an eye.

9. A medical treatment system comprising:
a medical treatment instrument;
a console configured to supply fluid to said medical treatment instrument in a defined operating mode of the medical treatment system;
a tube for feeding an irrigation fluid from said console to said medical treatment instrument and for discharging an aspiration fluid from said medical treatment instrument to said console;
said tube including a first channel for feeding an irrigation fluid from said console to said medical treatment instrument;
said tube further including a second channel for discharging the aspiration fluid from said medical treatment instrument to said console;
said first channel and said second channel being arranged in parallel next to each other in a direction of longitudinal extent of the tube;
said first channel having a first wall including a first material having a first hardness;
said second channel having a second wall including a second material having a second hardness;
said second hardness being greater than said first hardness;
said tube being formed from two individual tube elements including a first tube element for said first channel and a second tube element for said second channel;
said first tube element and said second tube element being welded or adhesively bonded in a connection region; and,
wherein a difference in hardness between said first hardness and said second hardness is at least 10 ShA and at most 15 ShA.

10. The medical treatment system of claim 9, wherein the medical treatment system is an ophthalmosurgical system for treating an eye.
